(19) United States
(12) Patent Application Publication
(10) Pub. No.: US 2025/0368989 A1
(43) Pub. Date: Dec. 4, 2025

(54) NON-CODING RNA SEQUENCES CAPABLE OF INCREASING THE EXPRESSION OF CHD8 AND CHD2 PROTEINS

(71) Applicants: UNIVERSITA' DEGLI STUDI DI TRENTO, Trento (IT); FONDAZIONE ISTITUTO ITALIANO DI TECNOLOGIA, Genova (IT)

(72) Inventors: Marta Biagioli, Trento (IT); Francesca Di Leva, Trento (IT); Michele Arnoldi, Trento (IT); Stefano Gustincich, Genova (IT)

(21) Appl. No.: 18/869,969

(22) PCT Filed: May 23, 2023
(86) PCT No.: PCT/IT2023/050128

§ 371 Date: Nov. 27, 2024

(30) Foreign Application Priority Data
Jun. 1, 2022 (IT) 102022000011546

(51) Int. Cl. C12N 15/113

(52) U.S. Cl. CPC C12N 15/113

(57) ABSTRACT

The present invention relates to some RNA sequences which have been found effective in the treatment of a neurological disorder. Specifically, the neurological disorder is an autism spectrum disorder or autism and/or epilepsy.

The present invention relates to the field of neurological disorders, in particular it relates to autism spectrum disorders (or autism) and epilepsy. Specifically, the invention relates to non-coding RNA sequences which have been shown to be able to increase in a specifical and controlled way the expression of the CHD8 and CHD2 proteins.

### State of Art

Autism spectrum disorders (ASD) and epilepsy refer to a group of complex developmental brain disorders.

Autism is generally characterized by difficulties in social interaction, verbal and non-verbal communication, repetitive behaviours. It is a fairly common disorder, affecting 1 in 68 children (more than 2 million individuals in the United States and tens of millions worldwide) with troubling recent statistics suggesting that prevalence rates have been increasing in recent years.

Epilepsy is characterized by recurring seizures, usually caused by abnormal neuronal activity. There are about 50 million people who live with epilepsy with a prevalence between 4 and 10 people per 1000.

Currently, there are no definitive medical treatments for these neurological disorders, except for a few symptomatic drugs, generally with little efficacy, mainly aimed at alleviating the behavioural symptoms associated with autism and epilepsy.

Over the past five years, scientists have identified a number of rare genetic changes, called mutations, associated with autism and epilepsy. In particular, two genes, CHD8 and CHD2, have been mutated in independent studies and, therefore, are among the main risk factors. The two factors, CHD8 and CHD2, belong to the same protein family of chromodomain helicases, capable of binding to DNA and regulating chromatin compaction and accessibility. Most of the mutations in CHD8 and CHD2 identified so far are inactivating mutations, i.e. they cause a reduction in the expression levels of the protein or its functionality. A class of non-coding RNAs capable of increasing, in a specific and controlled way, the expression of target proteins is known. The activity of these non-coding RNAs, naturally occurring in both mice and humans, is based on two functional elements (see FIG. 1 below): a binding domain which mediates binding to the target transcript coding for the protein of interest and an effector domain which, thanks to the presence of repeated sequences (SINE, Alu or FRAM elements), mediates binding to heavy polysomes, inducing an increase in protein synthesis of the target protein. The discovery of this new class of non-coding RNAs laid the foundations for the development of the technology, SINEUP, which aims to recover the phenotypes associated with the reduction of functional protein levels, resulting from inactivating mutations. SINEUP has been successfully tested in various mouse and human cell models to induce an upregulation of proteins linked to pathologies generated by haploinsufficiency [DJ-1 in Parkinson's disease; FXN, in Friedereich's ataxia] and also in in vivo models which are both aquatic [Cox7, in microphthalmia with linear skin defects, using the ‘medaka fish’] and murine [increased expression of Gdnf protein (reduced in several neurodegenerative pathologies such as Parkinson's disease) after viral injection into the striatum of adult mice].

Although research has identified hundreds of genes that are risk factors for autism, each of these variants is present in only a very small percentage of individuals with the disease. Furthermore, in many patients, rather than arriving at the identification of a single cause (mutation in a single gene) associated with the disease, a complex combination of different genetic factors is found that influence early brain development. Therefore, although potentially functional, in light of what has been illustrated, this technology has the limit of being useful for a small number of individuals. However, the fact of being able to easily tailor the binding domain of SINEUP to a specific gene of interest represents an important feature for the development of a personalized therapy.

As a further consideration, the considered pathologies are disorders affecting the correct development of the central nervous system, already during the phases of embryonic development and gestation in utero. It is understandable that it would be ideal to be able to intervene with a treatment already during these early stages of embryonic development in order to minimize the damage due to the scarcity of the proteins in question. However, it is also understandable that the use of any experimental therapeutic technology to be administered in utero is associated with extremely important ethical issues, especially if one considers that the definitive diagnostic test of autism spectrum disease and epilepsy is defined well after the birth of children, around 18-24 months of life.

Therefore, the need is felt to find a solution which allows the above problems of the prior art to be overcome. In particular, we are considering taking a cue from Rett syndrome (caused by mutations causing haploinsufficiency of the MeCP2 protein) for which, in preclinical studies, even treatments aimed at reactivating the defective gene after the birth of the individual have shown to be effective to prevent further deterioration of symptoms (Guy et al. 2007). In agreement with this hypothesis, a recent study demonstrates that CHD8 is also fundamental in mouse models not only during the embryonic period of brain development, but also in the weeks immediately following birth. Therefore, it is conceivable that, even the administration of SINEUP in early periods, but following the birth of the individual, may prove effective in recovering a series of symptoms and therefore in improving the quality of life of patients affected by these mutations.

### SUMMARY OF THE INVENTION

The Applicants have now found an approach based on the use of RNA sequences (SINEUP) aimed at the specific risk factors of the neurological diseases of interest. Starting from these two specific risk factors of autism spectrum disorders (ASD) and epilepsy, in particular the inactivating mutations in CHD8 and CHD2, the non-coding RNA sequences identified have been shown to be able to specifically and controlled the expression of CHD8 and CHD2 proteins.

Therefore, a first object of the present invention relates to RNA sequences according to claim 1.

Advantageously, using the technology based on SINEUP, the stimulation of protein production is obtained within a physiological range and only in the cells/districts of the body in which the transcript of interest is usually expressed. Furthermore, the proposed technology allows the increase of protein expression only when it is necessary for the cells/district of interest, i.e. when the target mRNA is naturally transcribed. Another advantage of the invention is that of allowing the recovery of the levels and functionality of the aforementioned target proteins, without altering the DNA sequence of the individual, but only by acting on the RNA molecule.

Further features and advantages of the disclosure of the invention will become apparent from the description of embodiments of the invention, given as an indication of the invention.

### BRIEF DESCRIPTION OF THE FIGURES

FIG. 1: Schematic representation of the SINEUP-CHD8 molecules.

FIG. 2: Effect of CHD8 translational enhancement when SINEUP-CHD8 is administered to a model of hiNPC-GM8330 neural progenitors exhibiting reduced CHD8 levels.

FIG. 3: Administration of SINEUP_CHD8_001 and SINEUP_CHD8_003 in the CHD8 haploinsufficiency model, recovers molecular phenotypes [(transcription of target genes (SHANK3, MBD3) and altered deposition of a specific histone modification (H3K36me3)] due to reduction of the functional protein.

FIG. 4: The administration of SINEUP-CHD8 is efficient in recovering the defective levels of CHD8 protein in human fibroblast lines obtained from patients carrying CHD8 mutations.

FIG. 5: Administration of SINEUP_chd8 in aquatic model ‘zebrafish’ with reduced levels of CHD8, recovers the phenotype of macrocephaly (excessive head development) in animals.

FIG. 6: SINEUP_CHD2 administration increases CHD2 protein levels in an in vitro human induced pluripotent cell model of CHD2 haploinsufficiency.

### DETAILED DESCRIPTION OF THE INVENTION

For the purposes of the invention, definitions of some terms used in the present description and in the attached claims are provided below.

The RNA sequences of the present invention, i.e. SINEUP_CHD8_001 having nucleotide sequence GCCATCTTGGGAAAGTAATGGAGGGTACTTCTCCAAGGTCTAGG,
SINEUP CHD8 003 having nucleotide sequence CCATTATCTGAGCCTGCTGTACAGAGGACAGAGTCACTGGCTGG,
SINEUP CHD2_006 having nucleotide sequence TCATCTTTTAATTCTTAAATATTAAGGGGGAGGGGGAATCTGTG e
SINEUP_CHD2_007 having nucleotide sequence ACATCTTCTTCACATCAGCTATCCGTTCCTTCTTAGAGGCTGGC, as understood herein, are RNA sequences, in particular non-coding RNA sequences, capable of increasing in a specifical and controlled way the expression of the CHD8 and CHD2 proteins. It has already been shown in WO 2012/133947 that the target RNA binding sequence needs to have at least 60% homology with the transcript of interest, however, the higher the homology with the transcript of interest, the higher the functional possibilities of the molecule.

In particular, the sequences SINEUP_CHD8_001 and SINEUP_CHD2_006 have: 1. Complementary and inverted sequence to a stretch of the non-coding region, corresponding to 40 nucleotides upstream of the translation start site (AUG-1Met); 2. Complementary and inverted sequence to a segment of the coding region corresponding to 4 nucleotides downstream of the translation start site (AUG-1Met) [sequence defined overall-40/+4] for the short and long isoforms of CHD8 and CHD2.

The sequence SINEUP_CHD8_003 has: 1. Complementary and inverted sequence to a stretch of the coding region, corresponding to 40 nucleotides upstream of the fifth internal methionine in the NM_001170629 isoform and 40 nucleotides upstream of the second internal methionine in the NM_020920 isoform, in position 384, on exon 3 and in position 105 on exon 3 respectively for the two isoforms of CHD8 (AUG-5Met, AUG-2Met); 2. Complementary and inverted sequence of a segment of the coding region corresponding to 4 nucleotides downstream of the fifth and second internal methionine respectively for the isoform NM_001170629 and NM_020920 of CHD8 (AUG-5Met and AUG, 2Met) [sequence defined overall-40/+4]. The sequence SINEUP_CHD2_007 presents: 1. Complementary and inverted sequence to a stretch of the coding region, corresponding to 40 nucleotides upstream of the third internal methionine, in position 99, on exon 4 (AUG, 3Met); 2. Complementary and inverted sequence of a segment of the coding region corresponding to 4 nucleotides downstream of the third internal methionine (AUG-3Met) [sequence defined overall-40/+4] for the NM_001271 and NM 001042572 isoforms of CHD2.

As extensively reported above, the CHD8 and CHD2 proteins represent two specific risk factors of autism spectrum disorders.

The present invention therefore relates to an RNA sequence selected from
SINEUP_CHD8 001 (SINEUP_001) having nucleotide sequence GCCATCTTGGGAAAGTAATGGAGGGTACTTCTCCAAGGTCTAGG,
SINEUP_CHD8_003 (SINEUP_003) having nucleotide sequence CCATTATCTGAGCCTGCTGTACAGAGGACAGAGTCACTGGCTGG,
SINEUP CHD2_006 (SINEUP_006) having nucleotide sequence TCATCTTTTAATTCTTAAATATTAAGGGGGAGGGGGAATCTGTG, SINEUP_CHD2_007
(SINEUP_007) having nucleotide sequence ACATCTTCTTCACATCAGCTATCCGTTCCTTCTTAGAGGCTGGC or a combination thereof.

In a preferred embodiment, the SINEUP_CHD8_003 molecule appears to show the best functional characteristics. It is possible that the combination of two or more of the reported RNA sequences (for example SINEUP_CHD8_001+SINEUP_CHD8_003, or SINEUP_CHD2_006+SINEUP_CHD2_007, or SINEUP_CHD8_001/003 and SINEUP_CHD2_006/007) could be an improvement.

According to a preferred aspect, the RNA sequence of the present invention is embedded inside an expression vector. Expression vectors which can be used are known to those skilled in the art. For example:
1. Plasmid pCDNA 3.1 (−), CMV promoter, BGH terminator polyA (+)
2. pDUAL-EGP, H1 promoter, BGH polyA (+) terminator
3. Lentivirus pAIB (third generation), SSFV promoter

The above sequences can be included in a pharmaceutical composition. Said pharmaceutical composition may further comprise at least one pharmaceutically acceptable excipient.

The present invention also relates to compositions comprising the aforementioned molecules of nucleic acids or DNA. Any composition is included allowing to deliver said functional nucleic acid molecules by viral vectors (AAV, lentivirus or the like), and non-viral vectors (nanoparticles, lipid particles or the like).

A further object of the present invention relates to a pharmaceutical composition comprising at least one RNA sequence selected from among SINEUP_CHD8_001, SINEUP_CHD8_003, SINEUP_CHD2_006 and SINEUP_CHD2_007. Said pharmaceutical composition may further comprise at least one pharmaceutically acceptable excipient.

A further object relates to an RNA sequence selected from SINEUP_CHD8_001, SINEUP_CHD8_003, SINEUP_CHD2_006, SINEUP_CHD2_007, or a combination thereof, or of the pharmaceutical composition comprising it, for use in the treatment of a neurological disorder, preferably, wherein said neurological disorder is autism spectrum disorder or autism and/or epilepsy.

A further object is an RNA sequence selected from SINEUP_CHD8_001, SINEUP_CHD8_003, SINEUP_CHD2_006, SINEUP_CHD2_007 or a combination thereof, or of the pharmaceutical composition comprising it, for use in a method of prevention of a neurological disorder, preferably, in which said neurological disorder is autism spectrum disorder or autism and/or epilepsy.

A further object is an RNA sequence selected from SINEUP_CHD8_001, SINEUP_CHD8_003, SINEUP_CHD2_006, SINEUP_CHD2_007 or a combination thereof, or a pharmaceutical composition comprising it, for use in a method aimed at increasing the expression of CHD8 and CHD2 proteins.

What is reported in the present document is to be understood as a simplification and not a limitation. Furthermore, the person skilled in the art will be able to understand that modifications can be made without departing from the scope of the present application.

### Examples

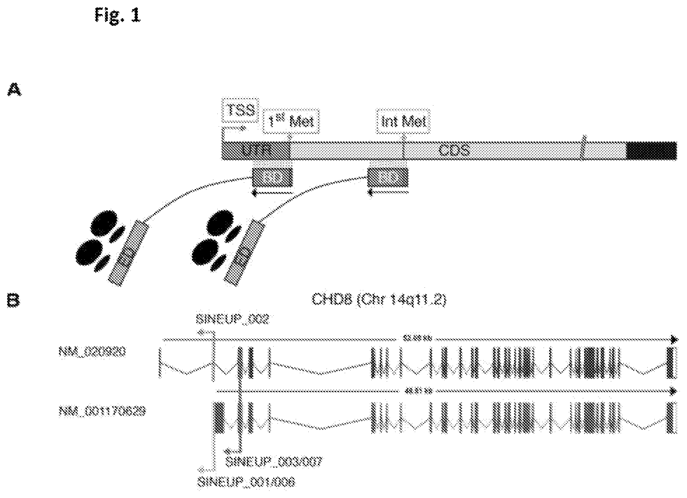

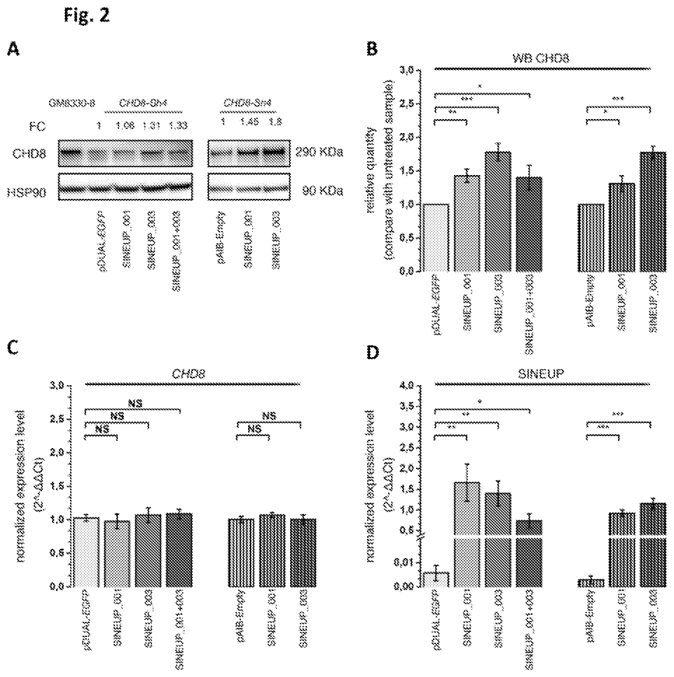

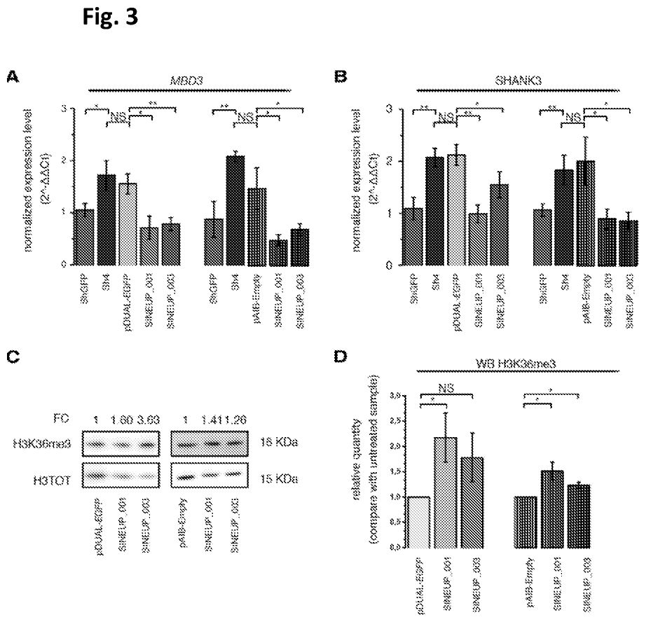

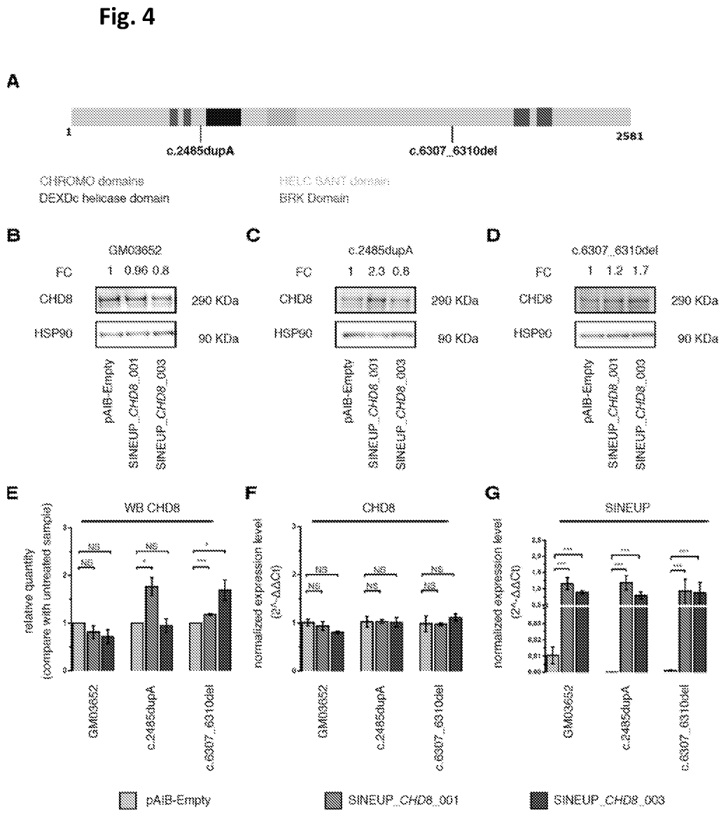

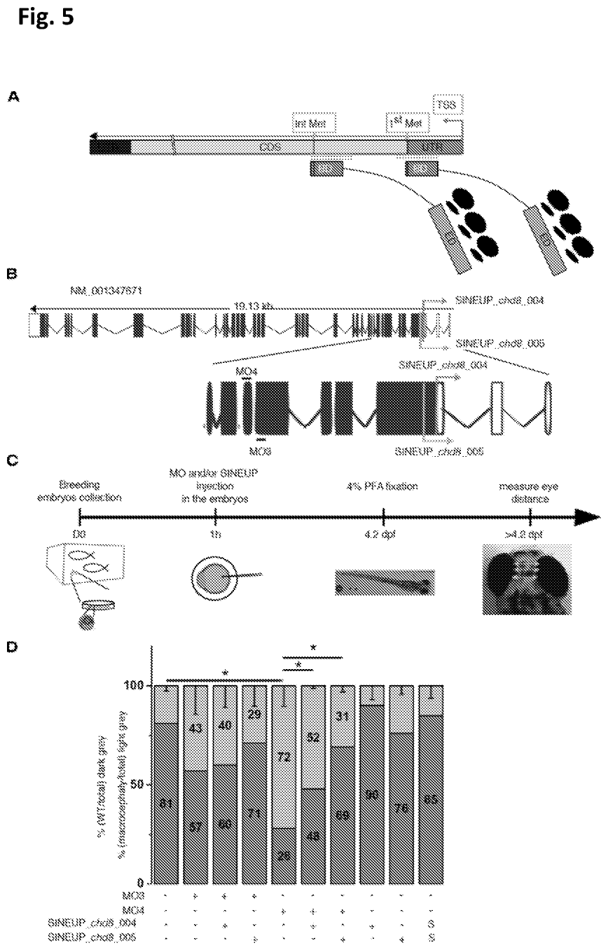

EXAMPLE 1, related to FIG. 1. In order to identify the most efficient SINEUP molecules, we analyzed the transcripts encoded by CHD8 in humans. We identified two transcripts, NM_001170629 and NM_020920 (see FIG. 1A.). Therefore, referring to the literature and considering that SINEUP is composed of a binding domain (DL in blue), which overlaps, with an antisense orientation, the mRNA encoding the protein of interest (here CHD8) and from an effector domain (DE in green) that does not overlap with the transcript of interest (FIG. 1A.), we drew 3 binding domains (DL). SINEUP DL confers specificity to the molecule while SINEUP DE can recruit the target mRNA and place it on the polysomes to regulate their protein synthesis. Therefore, three SINEUP molecules were designed which respectively recognize the translation start site (1Met) and an internal methionine in the coding sequence (Met Int). In FIG. 1A, the structural elements of protein-coding mRNA are shown: transcription start site (SIT), 5′-untranslated region (RNC, blue), coding sequence (SC, light blue), and 3′-untranslated region translated (RNC, dark blue). FIG. 1B, on the other hand, shows a schematic representation of SINEUP-CHD8 (SINEUP_001/_003) which identifies their position within the human isoforms of CHD8, respectively NM_020920 and NM_001170629. SINEUP_001 targets the translation initiation site (first methionine) of isoform NM_001170629, SINEUP_002 targets the first methionine of isoform NM_020920, while SINEUP_003 recognizes an internal methionine, common to both isoforms. SINEUP_001/002/003 have a canonical length of 44 nucleotides (see detailed description of the invention). Once designed, the SINEUP molecules were inserted/cloned into expression and viral vectors (see detailed description of the invention) to then proceed to their transfer into the cells of interest.
EXAMPLE 2, relating to FIG. 2. Once the design of the SINEUP_CHD8 molecules in example 1 was completed, specific primers were ordered in order to proceed with the insertion of the sequences into expression and viral vectors. Subsequently, in order to test the efficiency of SINEUP_CHD8, cells showing reduced levels of CHD8 were used (these models were obtained by using “short hairpin-Sh” CHD8-Sh4). Cells with reduced protein expression were treated by electroporation or infected with lentiviruses, with the control vector alone (pDUAL EGFP for electroporation and pAIB Empty for lentivirus infections) or with the different SINEUP vectors aimed at recognizing CHD8 (SINEUP_001-003). In the case of the electroporation experiments, the combined administration of SINEUP_001 with SINEUP_003 (SINEUP_001+003) was also carried out in the same quantities. The results of these experiments were analysed by quantifying proteins (western blot technique, WB) and RNAs (quantitative PCR technique, RT-qPCR). As can be seen from the images shown in FIG. 2A., the expression of SINEUP_001, 003 or 001+003 is able to recover the correct expression of the protein as revealed by the experiments of WB (FIG. 2A.) and the bar graph (FIG. 2B.) reporting the quantification of the change in CHD8 protein expression levels from WB experiments (A). HSP90 was used as a loading control. As expected, the change in CHD8 protein amounts is not accompanied by transcriptional alterations, quantified by RT-qPCR of CHD8 transcript expression after exposure to 001 (light gray), 003 (gray), 001+003 (dark gray) or to the control vector (lighter gray) (FIG. 2C.). These positive results agree both if obtained by electroporation (bars on the left in 2B and 2C) and by administration of lentiviral vectors (bars on the right with lines in 2B and 2C). Finally, FIG. 2D. shows RT-qPCR expression of SINEUP 001, 003, 001+003 molecules or control vectors, which, as expected, are elevated when the SINEUP molecule is present. TBP and NONO were used as stable transcripts for the quantification of experiments. For statistical analysis, Welch-corrected t-tests were performed. NS, Not Significant, P>0.05, * P<0.05, ** P≤0.01, P≤0.001. n=8-14 experiments per condition.
EXAMPLE 3, related to FIG. 3. To study the functionality of the CHD8 proteins produced in Example 2 through the overexpression of SINEUP both by treating the cells with electroporation and with lentiviral vectors, we evaluated the efficacy in recovering altered phenotypes. In particular, due to the reduction of the amount of CHD8 in CHD8-Sh4 cells, there is an alteration of the transcription of target genes (among which SHANK3 and MBD3) and an altered deposition of a specific histone modification (H3K36me3)). The results of these experiments were analysed by quantifying the RNAs (quantitative PCR technique, RT-qPCR) and the proteins (western blot technique, WB) as can be seen from the images shown in FIGS. 3A. and 3B. first, the expression of the two transcripts was evaluated in cells with reduced amount of CHD8 (Sh4 in dark grey) compared to the control (ShGFP control line in green with amount of physiological CHD8). As reported in the figure, both SHANK3 and MBD3 are more expressed when CHD8 is reduced (Sh4 line) than in the control (ShGFP line). When the control vectors were administered (pDUAL EGFP in the case of the electroporation and pAIB Empty in the case of the experiments with the lighter grey lentiviral vectors), no perturbation in the expression levels of the two transcripts was observed compared to the untreated line Sh4. In contrast, the expression of SINEUP_001 (light grey) or 003 (grey) is able to recover the correct expression of the SHANK3 and MBD3 transcripts as revealed by RT-qPCR experiments (FIG. 3A. SHANK3 and FIG. 3B. MBD3). TBP and NONO were used as stable transcripts for the quantification of experiments. Finally, FIGS. 3C. and 3D. show the ability of the proteins produced in example 2 through the administration of SINEUP_001 and 003 to restore the deposition of the histone modification H3K36me3 by quantification by western blot (WB). The total amount of histone H3 was used as a normalizer. These positive results were consistent both when obtained by electroporation (left bars in 3A, 3B and 3D) and by administration of lentiviral vectors (right bars lined in 3A, 3B and 3D). For statistical analysis, Welch-corrected t-tests were performed. NS, Not Significant, P>0.05, * P<0.05, ** P<0.01, *** P<0.001. n=3-7 experiments per condition.
EXAMPLE 4, related to FIG. 4. In order to evaluate the efficacy of SINEUP with respect to CHD8 in different cellular models, we treated primary fibroblasts derived from patients presenting inactivating mutations in the coding sequence of CHD8. As can be seen from FIG. 4A. the two patient lines respectively have a duplication of nucleotide 2485 (c.2485dupA) and a deletion of 3 nucleotides at position 6307-6310 (c.6307_6310del). Such mutations modify the coding sequence by creating a premature stop codon which results in the formation of a truncated, possibly non-functional protein. Major domains of CHD8 are represented as coloured boxes along the CHD8 protein structure in grey (green, CHROMO, chromodomain; blue, DEXDc, helicase domain; light green, HELC, SANT domain; brown, BRK domains of unknown function). The control fibroblasts (GM03652 with physiological amount of CHD8) and those obtained from patients carrying mutations on CHD8 (who have a reduced amount of CHD8), were treated by lentiviral transduction (as in example 2) with only the control vector (pAIB-Empty in light grey) or the different molecules of SINEUP_001 (grey), or 003 (dark grey). The results of these experiments were analysed by quantifying proteins (western blot technique, WB) and RNAs (quantitative PCR technique, RT-qPCR). As can be seen from the images shown in FIG. 4B. and 4E. the expression of SINEUP_001, or 003 is unable to increase the expression of the protein when it is present in physiological amounts (control line GM03652) as revealed by the experiments of WB (FIG. 4B.) and the bar graph (FIG. 4E.) reporting the quantification of the change in CHD8 protein expression levels in WB experiments (B). However, the administration of the same SINEUPs is able to increase the expression of the protein in patient cells that have a reduced amount of CHD8 as can be seen from the experiments of WB (FIGS. 4C. 4D, and 4E, samples c.2485dupA and c. 6307_6310del). HSP90 was used as a loading control. As expected, the change in CHD8 protein amounts was not accompanied by transcriptional alterations, quantified by RT-qPCR of CHD8 transcript expression after exposure to 001 (grey), 003 (dark grey), or control vector (clear grey)) (FIG. 4F.). Furthermore, there is no evidence of a change in the amount of CHD8 expressed in the GM03652 control line when SINEUP is present (FIG. 4F). Finally, FIG. 4G. shows how, by RT-qPCR, the molecules of SINEUP 001, 003, or of the control vectors, show high levels of expression. TBP and NONO were used as stable transcripts for the quantification of experiments. For statistical analysis, Welch-corrected t-tests were performed. NS, Not Significant, P>0.05, * P≤0.05, ** P<0.01, *** P≤0.001. n=3-5.
EXAMPLE 5, related to FIG. 5. In order to evaluate the ability of the SINEUP molecules to increase the production of the chd8 protein also in in vivo models, we have chosen the zebrafish. Previously the ability of SINEUP to increase the translation of target transcripts in a similar fish, the Medaka fish, had been demonstrated. In our case, we selected zebrafish because models of this fish were available in the scientific community in which morpholino molecules were used to reduce chd8 expression by about 50%, mimicking the human pathological model. Only one transcript of chd8 (NM_001347671) is present in zebrafish. FIG. 5A. shows a representation of the modular structure of the SINEUPs drawn in the aquatic model. Structural elements of protein-coding mRNA are shown: transcription start site (SIT), 5′-untranslated region (RNC, blue), coding sequence (SC, light blue), and 3′-untranslated region (RNC, dark blue). The figure also shows the SINEUP molecule where the effector domain (DE in green) which does not overlap with the transcript of interest and the binding domain (DL in blue) which recognizes the transcript of interest can be distinguished. We designed 2 binding domains (DL) SINEUP_004 and SINEUP_005 which recognize, respectively, the first methionine (1 Met) and the internal methionine (int Met) of the fish chd8 isoform NM 001347671. The rationale used for the design was the same as that used for the SINEUPs recognizing CHD8 in humans, i.e. 44 nucleotides with −40/+4 conformation with respect to the Methionine of interest. Primers identifying binding domains for SINEUP molecules used in zebrafish were synthesized and cloned/inserted into an expression vector upstream of the effector domain. FIG. 5B shows the schematic representation of the chd8 transcript NM_001347671 and the location of SINEUP_004 and 005 in exon 3 of the transcript. The enlargement in FIG. 5B also shows the positions recognized by the morpholino molecules which artificially reduce the level of chd8 in zebrafish: In particular, MO3 targeting exon 7 of NM_001347671 and MO4 targeting exon 8 of NM_001347671 were used. same transcript. FIG. 5C shows the graphical representation of the experimental design. Zebrafish, strain Tu/Tu or Tu/ab, are kept in an enclosure under controlled water, temperature and light/dark conditions. The day before the experiment the male fishes are separated from the female ones by means of a separator which is removed on the morning of day 0 (DO, in the graphical representation). As soon as the eggs are laid, they are injected with morpholino MO3, MO4 or control morpholino and/or with SINEUP_004/SINEUP_005. The injections are made directly into the nucleus of the embryo immediately after spawning and in any case before it separates at the 4-cell level, thus allowing the diffusion of the molecules throughout the fish during subsequent development. Fish embryos are kept in an incubator for 4.2 days after fertilization. At day 4.2 the fish are fixed in 4% paraformaldehyde and the distance between the eyes is measured and used as an indicator of macrocephaly. FIG. 5D demonstrates the ability of SINEUP_004 and 005 to recover from macrocephaly induced by morpholinos that reduce the level of chd8. The results are reported by bar graphs indicating the percentage of zebrafish that show macrocephaly (light grey) or have normal head size (dark grey). A fish showing an increase in eye distance>12% of the mean eye distance of normal fish of the same strain is considered macrocephalic (Sugathan et al. 2014). Administration of MO3 or MO4 leads to an increase in the percentage of macrocephalic fish to 43% and 78% compared to the 18% observed in healthy fish (columns 2, 5 and 1, respectively). Administration of SINEUP 004 or 005 is unable to reduce MO3-induced macrocephaly (columns 2, 5, 3 and 4, respectively).

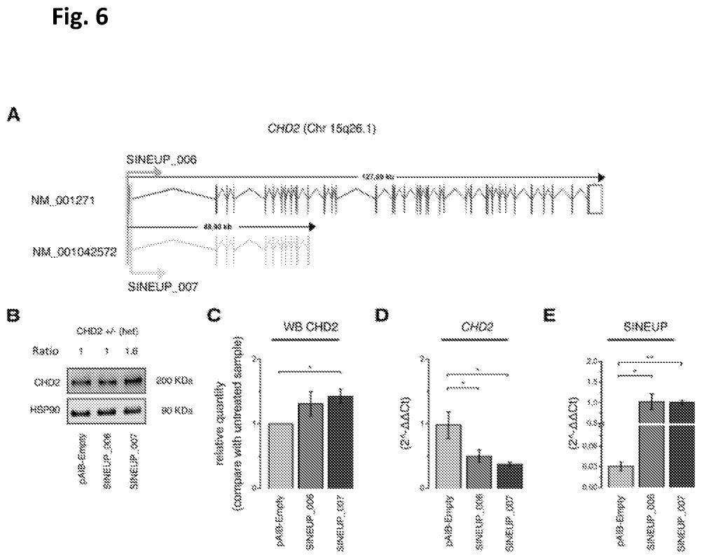

In contrast, administration of SINEUP_004 is able to significantly reduce MO4-induced macrocephaly from 72% to 52% (columns 6 and 5), and SINEUP_005 is able to significantly reduce MO4-induced macrocephaly from 72% to 31% (columns 5 and 7). Furthermore, we observe that, as desirable, the administration of SINEUP_004, 005 alone or of a morpholino with an a-specific sequence does not induce macrocephaly (columns 8, 9 and 10). The results in zebrafish confirm those obtained in human models and show a greater efficacy of SINEUP_005 directed towards internal methionine in reducing artificially induced macrocephaly with the use of morpholines. n≥25 embryos/conditions. P>0.05, * P≤0.05.
EXAMPLE 6. Similarly to what is reported in example 1., also for CHD2 we analysed the structure of transcripts in humans. Again, we identified two transcripts, NM_001271 and NM_020920 (see FIG. 6A.). Again, we proceeded to design two SINEUP-CHD2 molecules (SINEUP_006/007). In FIG. 6A. their position is described in relation to human isoforms of SINEUP_006 targets the translation initiation site (1 Met) while SINEUP_007 recognizes an internal methionine, common to both isoforms. SINEUP_006/007 have a canonical length of 44 nucleotides (see detailed description of the invention). Once the design of the SINEUP_CHD2 molecules was completed, also in this case, specific primers were ordered in order to proceed with the insertion of the sequences into viral vectors. Subsequently, in order to test the efficiency of SINEUP_CHD2, a model of CHD2 haploinsufficiency (reduced transcript and protein expression) was created using the CRISPR-Cas9 technique. The activity of CRISPR-Cas9 proved to be able to reduce the expression of the CHD2 gene (CHD2+/−) to a value of about 50% of expression (both RNA and protein), compared to the control line. Human induced pluripotent cells (iPS) with reduced expression of the protein (CHD2+/−) were treated with the control vector alone (pAIB Empty) or with the different SINEUP vectors targeting CHD2 recognition (SINEUP_006/007). The results of these experiments were analysed by quantifying proteins (western blot technique, WB) and RNAs (quantitative PCR technique, RT-qPCR). As can be seen from the images shown in FIG. 6B. the expression of SINEUP_006-007 is able to recover the correct expression of the protein as revealed by the experiments of WB (FIG. 6B.) and the bar graph (FIG. 6C.) reporting the quantification of the change of the expression levels of the CHD2 protein in WB experiments (B). HSP90 was used as a loading control. As expected, the change in the amount of CHD2 protein is not accompanied by a transcriptional increase (indeed, in these experiments the CHD2 levels are reduced), quantified by RT-qPCR of the expression of the CHD2 transcript after exposure to 001 (grey), 003 (dark grey) or the control vector (lighter grey) (FIG. 6D.). Finally, FIG. 6E. shows RT-qPCR expression of SINEUP_CHD2_006, 007 molecules or control vectors, which, as expected, are elevated when SINEUP is present. TBP and NONO were used as stable transcripts for the quantification of experiments. For statistical analysis, Welch-corrected t-tests were performed. NS, Not Significant, P>0.05, * P≤0.05, ** P<0.01, *** P<0.001. n=3 experiment per condition.

### Experimental Part

### 1. Cell Lines

Human neuroprogenitor (hiNPC) lines were derived from induced pluripotent stem cells. The GM8330-8, Sh4-CHD8 and Sh-GFP lines were generated thanks to the use of shRNA directed respectively towards the coding sequences of the CHD8 and GFP genes and were kindly provided by the laboratory of Dr. Stephen Haggarty (Massachusetts General Hospital and Harvard Medical School, Boston, MA, USA).

Neuroprogenitors were grown on cell culture plates coated with poly-L-ornithine hydrobromide (20 μg/mL) and laminin (3 μg/mL) using the culture medium composed of DMEM, 30% v/v HAMF12, B27 2% v/v and a 1% v/v penicillin/streptomycin solution. The medium was supplemented with EGF (20 ng/ml), bFGF (20 ng/mL) and heparin (5 μg/mL). The cells were maintained in culture, in monolayer and in semi-confluent concentration, in a humidified incubator at 37° C. and 5% CO2

Fibroblasts originating from healthy individuals, GM03652 were kindly provided by the laboratory of Dr. Gemma Louise Carvill (Northwestern University, Feinberg School of Medicine, Chicago, IL, U.S.A.). Patient-derived fibroblasts TR0000002 (c.6307_6310del) and TR0000028 (c.2485dupA) which contain de novo mutations in the CHD8 gene, were kindly provided by the laboratory of Dr. Raphael Bernier (UW Autism Center, University of Washington, Seattle, WA, USA). Human fibroblasts were cultured in DMEM supplemented with 10% v/v FBS, 1% v/v L-glutamine, and 1% v/v penicillin-streptomycin solution. The fibroblasts were maintained in culture, in monolayer and in semi-confluent concentration, in a humidified incubator at 37° C. and 5% CO2. Human kidney cells, HEK293T, were maintained in culture under the same conditions described for fibroblasts.

### 2. Design and Cloning of SINEUP Molecules Directed Towards the CHD8 Transcript

The SINEUP molecules directed towards the human CHD8 gene and the zebrafish chd8 gene were cloned respectively into the pDUAL_EGFP and pCS2 plasmid vectors which already contained the inverted SINEB2 nucleotide sequences (i.e. ED). Sequences recognizing CHD8/chd8 were selected in the −40/+4 regions overlapping either the translation start site or an internal methionine. In particular, SINEUP_CHD8_001 is drawn on the human CHD8 transcript sequence identified with NM_001170629, SINEUP_CHD8_002 is drawn on the human CHD8 transcript sequence identified with NM_001170629, SINEUP_CHD8 003 is drawn on the internal methionine present in both CHD8 transcripts), SINEUP_chd8_004 and SINE UP chd8 005 are drawn on the zebrafish transcript sequence of chd8 identified with NM 001347671. The selected sequences were synthesized and cloned with inverted orientation into the respective plasmid vector, upstream of the ED sequences, using a T4 ligase enzyme. The vectors containing the SINEUP sequences were produced in sufficient quantities using commercial plasmid maxiprep kits. The secondary structures formed by the SINEUP molecule were predicted using the RNA-FOLD Web server software (http://rna.tbi.univie.ac.at/cgi-bin/RNAWebSuite/RNAfold.cgi) while the site analysis transcription initiation of the CHD8 gene was done using the ZENBU software (https://fantom.gsc.riken.jp/zenbu/). The analysis of the specificity of the molecules for the recognition of the CHD8/chd8 gene was done using the BLASTN software (https://blast.ncbi.nlm.nih.gov/Blast.cgi)

### 3. Production of Viral Molecules Containing SINEUP-CHD8

SINEUP_CHD8_001, SINEUP_CHD8_003 nucleic molecules were subcloned into the pAIB viral vector under the control of the SSFV promoter. Three plasmids were used for viral preparation: pAIB containing SINEUP of interest, psPAX2 for the viral genome and pHDMG VSV-G for the production of the protein envelope of the virus. The three plasmids were transformed into E. coli DH5a bacteria and cultured in LB broth containing antibiotic for selection at 37° C. Plasmids were purified using commercial kits and correct insertion of the SINEUP molecule was verified by restriction mapping and sequencing.

Lentiviral particles were produced in 40% confluent HEK293T cells, cultured in DMEM supplemented with 10% v/v FBS, 1% v/v L-glutamine, and 1% v/v penicillin-streptomycin solution. v. For each SINEUP a 1 ml solution was prepared containing Opti-mem (Gibco): 50 μl of PEI (Sigma), 10 μg of pAIB vector, 7.5 μg of psPAX2 vector and 2.5 μg of pHDMG vector. The solution was vortexed, allowed to incubate at room temperature for 10 minutes before adding to cultured cells. The cells were placed in an incubator at 37° C. and the day after the medium was changed with medium also containing the penicillin-streptomycin solution at 4% v/v. After 48 h the culture medium containing the lentiviral particles was collected and centrifuged at 500 g for 5 minutes. The supernatant was collected and filtered with a PES 0.40 filter and subsequently divided into ready-to-use aliquots. The infectious titer of lentiviruses was measured as reverse transcriptase units (RTU) by the SG-PERT method (Vermeire et al. 2012).

### 4. Cell Electroporation

The pDUAL_EGFP plasmid vector containing SINEUP_CHD8_001, SINEUP_CHD8_002 or SINEUP_CHD8_003 was transferred into hiNPC lines GM8330-8 or Sh4-CHD8 by electroporation using program A-033 of the Nucleofector instrument. 5 μg of plasmid were used which were electroporated into 5*10\hat{ }6 cells (hiNPC). The hiNPCs were cultured for 24 or 48 hours and then harvested for RNA and protein extraction.

### 5. Transduction of Viral Vectors

Patient-derived hiNPCs and fibroblasts were plated at 60% confluency in antibiotic-free medium. The cells were treated with 1.5 RTU of lentiviral vectors in the presence of 4 ng/ml of polybrene (Sigma). The medium was changed to complete medium containing 1% v/v penicillin-streptomycin solution one day later. After 48 h of incubation at 37° C., the cells were harvested and used for the extraction and quantification of proteins and RNA.

### 6. Animal Model of Zebrafish (Zebrafish), RNA Production of SINEUP and Injection of Morpholini, Measurement of the Distance Between the Eyes

Zebrafish (Danio rerio) were raised in a temperature and light/dark cycle-controlled aquarium (28° C.; 14/10-hour light/dark cycle). Tu/Tu or Ab/Tu strains were used for this study. The zebrafish larvae in the stages used (2 and 4.2 days after fertilization) are not able to feed themselves and, therefore, are not subject to Italian legislation (Legislative Decree nr. 26/2014).

Two different morpholino antisense oligonucleotides (MO), already published by (Bernier et al. 2014; Sugathan et al. 2014) were purchased from GENE TOOLS, Inc. (USA). The two morpholinos, chd8_MO3 (5′-GAGAATGGAATCATAACTTACTTGA-3′) and chd8_MO4 (5′-GCAAATGTGCAAGCAAGTAACACCT-3′), are directed against the splice site of exon 7 and 8, respectively. A morpholino that does not recognize any genetic sequence in Dario rerio (Scrambled MO; length 25 nucleotides) was used as a negative control. Morpholinos were maintained in aliquots with a concentration of 20 μg. Before use, the molecules were diluted in PhenolRed and water at different final doses (8 ng and 4 ng) to be subsequently tested to evaluate their efficacy and eventual toxicity. SINEUP_chd8_004 and SINEUP_chd8 005 RNAs were transcribed in vitro from the pCS2+plasmid using the mMessage mMachine SP6 kit. Briefly, 5 reactions were prepared with 1 μg of pCS2+plasmid containing the SINEUP sequences. The plasmid was linearized using the NOT1 restriction enzyme and transcription was done using the SP6 mMessage mMachine kit. RNA was purified using a filter device. Zebrafish embryos were injected at the 1-4 cell stage using 200 ng of SINEUP_chd8 molecules. The experiments were conducted by injecting only SINEUP or morpholini or a combination of the two molecules using the Eppendorf® Femtojet® microinjector. At the 4.2 day stage post fertilization, the embryos were fixed with 4% PFA overnight at 4° C. After 3 washes with 1×PBS, eye distance measurements were taken in 3 different head regions using Photoshop.

### 7. Inverse Transcription and Quantitative PCR

Total RNA was extracted from zebrafish cells and embryos using the TRIZOL reagent. To remove the DNA contamination, the RNA was treated with DNase while the RNase inhibitor SUPERase was used to prevent its degradation, finally the RNA was purified with the RNeasy Mini Kit. The extracted RNA was reverse transcribed using the iScript cDNA Synthesis Kit. For the characterization of SNPs (changes in a nucleotide base) in the 5′ untranslated region of the chd8 gene, the DNase I treated RNA was reverse transcribed as described above and amplified using mastermix green. The primers used for PCR amplification are chd8_Fw1 (5′-CACTGGATATCACTCTTTCTTTGC-3′) and chd8 Rv (5′-GTGGTGTGTCATCAAAGAGGTC-3′). The amplification product was purified and subjected to Sanger sequencing. For quantitative PCR (RT-qPCR) experiments, 1 μg of RNA was reverse transcribed, the cDNA was diluted 1:15 and amplified with the iTaq™ Universal SYBR® Green Supermix protocol. The human genes NONO and TBP were used as genes for normalization (HKG). The expression level of the mRNA of interest was calculated with the ΔΔCt method, evaluating the 2\hat{ }-ΔΔCt value.

### 8. Protein Extraction and Western Blot Analysis.

Total proteins were extracted from the cells using RIPA reagent to which protease and phosphatase inhibitors were added. Samples were sonicated using the Q700 instrument. After sonication the samples were centrifuged at 12,000 g for 20 minutes at 4° C. to remove the DNA pellet. Proteins were quantified by BCA assay.

To perform Western blot experiments, the protein samples were separated on 4-12% BIS_Tris gel and transferred onto Amersham™ Protran™ 0.45 μm nitrocellulose membrane. The membranes were blocked with 5% w/v skimmed milk powder (NFDM) and incubated with the following primary antibodies: anti-CHD8 1:1000 (Novus Biologicals, cat. 10060417), anti-HSP90, 1:5000 (Bioss, cat. BSM-51215M). Proteins were identified using horseradish peroxidase conjugated secondary antibodies 1:10,000 anti-mouse IgG or 1:10,000 anti-rabbit IgG. The bands were visualized using ECL Select WB detection reagent. Signal quantification was performed with Imagelab software (BioRad).

To quantify H3K36me3, cells were resuspended in a hypotonic solution [10 mM Hepes pH 8, 10 mM KCl; 0.1 mM MgCl, 1 mM DTT] to which protease and phosphatase inhibitors have been added. After a brief incubation on ice, the cells were centrifuged at 5000 rpm for 10 minutes at 4° C. to remove the supernatant containing the cytosolic fraction. The nuclei in the pellet were resuspended in 0.2 N HCl, rotated overnight at 4° C. and centrifuged at 10,000 rpm for 10 minutes. The supernatant was collected and the proteins were quantified using the Bradford assay. For this type of experiment H3K36me3 (Abcam, #AB9050) and H3 (Cell Signaling Technology, #4499S) antibodies diluted 1:1000 in 5% NFDM were used. Western blot, band visualization and image acquisition were performed as previously described.

### 9. Statistical Analysis.

Statistical significance was assessed by Student's t-test for an average population or Welch's correction (Welch 1947). p<0.05 was considered as a significant value. All error bars represent the standard error of the mean (S.E.M). Fisher's test was used for experiments with zebrafish.

For clarity, among the sequences mentioned above, the sequence which is functional in inducing the protein increase is an RNA sequence, which derives from the corresponding DNA sequence, among those mentioned above.

The non-coding RNA molecule of the invention consists of two functional domains: the binding domain and the effector domain. The functionality of SINEUP is essential from these two domains, joined together to form a single molecule.

The binding and effector domains must be expressed together and contextually, via expression vectors and transfection or viral transduction. This point is also crucial: it is the over-expression of an artificial molecule that leads to an increase in the protein synthesis of the target protein, with a therapeutic effect;

The binding and binding domains, within the expression vector, must maintain a minimum distance, a specific detachment to allow the correct structural folding of the molecule and, therefore, its functionality.

Although not expressly reported here, the SINEUP RNA molecule can be modified in order to increase its functionality-methylation on m6A and pseudouridylation.

Further sequences have also been identified which, by targeting internal methionines, common to the two isoforms of CHD8 (and CHD2 in the other case), behave like SINEUP_003. All drawn sequences are 44 nucleotides (−40/+4 internal methionines).

It is not excluded that shorter sequences on the same regions may prove functional.

We report below these further sequences:

SINEUP_CHD8_010
CCAUUCCUGUCUUUCCCCCCGAAUGAGGAGCAGAGCUUGCUGGU,
SINEUP_CHD8_011
GCAUGACUUCCACAUCUGAAUUAUCAGAUGAGGUAUUACGUUUU,
SINEUP_CHD8_012
GCAUGGAAGGCAAAGUCUCGCCAUCUGGUUCUUGCACUGGUUCA,
SINEUP_CHD8_013
GCAUAGAAAGCACUUUGUCUACAAUGGCUGCAUCUUCUUCACUG,
SINEUP_CHD8_014
UCAUCUGAGCCAUUUUGGUUUUGAAGCGCUUUAAUUUUUGAUGU
AUCCUCUUA,
SINEUP_CHD8_015
UCAUUUCUGUCCAUGUAUUAAAUUCUCGCUCCCAGUUAGUAAUU,
SINEUP_CHD8_016
ACAUUUCAUACUGUUGAAUCAUCUGCCUGCUGGCCAGACUGCCA,
SINEUP_CHD8_018
GCAUCAUUGGCUUAAGAAUGGCCUGUAGCUUUUGAACCUGUUCC,
SINEUP_CHD8_019
CCAUCAUUGUGUUAAGUAGAUUAGGCAUGUUGGUAUGACCUGCC,
SINEUP_CHD2_010
UCAUUUCAAUGAGAUCAUCUGAGUCAGUCUCAAAGUCAUCAUCU,
SINEUP_CHD2_011
CCAUCCAUUUACAUAGAUAUUCGGGCUCAUUUGAGGGUGCCGGC,
SINEUP_CHD2_012
CCAUUUCAUCAGCAAGGAUUACACUAUUAUUUUUGCACCAGGAA,

SINEUP_CHD2_013 having nucleotide sequence UCAUCAUCUUUUAAUUCUUAAAUAUUAAGGGGGAGGGGGAAUCU.

Therefore, an object of the invention is an RNA or DNA sequence encoding an RNA sequence selected from:
SINEUP CHD8 003 having nucleotide sequence CCATTATCTGAGCCTGCTGTACAGAGGACAGAGTCACTGGCTGG,
SINEUP_CHD8_010 having nucleotide sequence CCAUUCCUGUCUUUCCCCCCGAAUGAGGAGCAGAGCUUGCUGGU,
SINEUP CHD8 011 having nucleotide sequence GCAUGACUUCCACAUCUGAAUUAUCAGAUGAGGUAUUACGUUUU,
SINEUP CHD8 012 having nucleotide sequence GCAUGGAAGGCAAAGUCUCGCCAUCUGGUUCUUGCACUGGUUCA,
SINEUP CHD8 013 having nucleotide sequence GCAUAGAAAGCACUUUGUCUACAAUGGCUGCAUCUUCUUCACUG,
SINEUP CHD8 014 having nucleotide sequence UCAUCUGAGCCAUUUUGGUUUUGAAGCGCUUUAAUUUUUGAUGUAUCCU CUUA,
SINEUP CHD8 015 having nucleotide sequence UCAUUUCUGUCCAUGUAUUAAAUUCUCGCUCCCAGUUAGUAAUU,
SINEUP CHD8 016 having nucleotide sequence ACAUUUCAUACUGUUGAAUCAUCUGCCUGCUGGCCAGACUGCCA,
SINEUP_CHD8 018 having nucleotide sequence GCAUCAUUGGCUUAAGAAUGGCCUGUAGCUUUUGAACCUGUUCC,
SINEUP CHD8 019 having nucleotide sequence CCAUCAUUGUGUUAAGUAGAUUAGGCAUGUUGGUAUGACCUGCC,
SINEUP CHD2 007 having nucleotide sequence ACATCTTCTTCACATCAGCTATCCGTTCCTTCTTAGAGGCTGGC,
SINEUP CHD2 010 having nucleotide sequence UCAUUUCAAUGAGAUCAUCUGAGUCAGUCUCAAAGUCAUCAUCU,
SINEUP CHD2 011 having nucleotide sequence CCAUCCAUUUACAUAGAUAUUCGGGCUCAUUUGAGGGUGCCGGC,
SINEUP_CHD2_012 having nucleotide sequence CCAUUUCAUCAGCAAGGAUUACACUAUUAUUUUUGCACCAGGAA,
SINEUP_CHD2_013 having nucleotide sequence UCAUCAUCUUUUAAUUCUUAAAUAUUAAGGGGGAGGGGGAAUCU, or a combination thereof.

According to a preferred embodiment, the aforementioned sequence is embedded inside an expression vector. Thus an other object is an RNA or DNA sequence encoding an RNA sequence selected from:
SINEUP CHD8 003 having nucleotide sequence CCATTATCTGAGCCTGCTGTACAGAGGACAGAGTCACTGGCTGG,
SINEUP CHD8 010 having nucleotide sequence CCAUUCCUGUCUUUCCCCCCGAAUGAGGAGCAGAGCUUGCUGGU,
SINEUP CHD8 011 having nucleotide sequence GCAUGACUUCCACAUCUGAAUUAUCAGAUGAGGUAUUACGUUUU,
SINEUP_CHD8_012 having nucleotide sequence GCAUGGAAGGCAAAGUCUCGCCAUCUGGUUCUUGCACUGGUUCA,
SINEUP CHD8 013 having nucleotide sequence GCAUAGAAAGCACUUUGUCUACAAUGGCUGCAUCUUCUUCACUG,
SINEUP CHD8 014 having nucleotide sequence UCAUCUGAGCCAUUUUGGUUUUGAAGCGCUUUAAUUUUUGAUGUAUCCU CUUA,
SINEUP_CHD8_015 having nucleotide sequence UCAUUUCUGUCCAUGUAUUAAAUUCUCGCUCCCAGUUAGUAAUU,
SINEUP CHD8 016 having nucleotide sequence ACAUUUCAUACUGUUGAAUCAUCUGCCUGCUGGCCAGACUGCCA,
SINEUP CHD8 018 having nucleotide sequence GCAUCAUUGGCUUAAGAAUGGCCUGUAGCUUUUGAACCUGUUCC,
SINEUP CHD8 019 having nucleotide sequence CCAUCAUUGUGUUAAGUAGAUUAGGCAUGUUGGUAUGACCUGCC,
SINEUP CHD2 007 having nucleotide sequence ACATCTTCTTCACATCAGCTATCCGTTCCTTCTTAGAGGCTGGC,
SINEUP CHD2 010 having nucleotide sequence UCAUUUCAAUGAGAUCAUCUGAGUCAGUCUCAAAGUCAUCAUCU,
SINEUP_CHD2_011 having nucleotide sequence CCAUCCAUUUACAUAGAUAUUCGGGCUCAUUUGAGGGUGCCGGC,
SINEUP_CHD2 012 having nucleotide sequence CCAUUUCAUCAGCAAGGAUUACACUAUUAUUUUUGCACCAGGAA,
SINEUP CHD2 013 having nucleotide sequence UCAUCAUCUUUUAAUUCUUAAAUAUUAAGGGGGAGGGGGAAUCU, or a combination thereof, wherein said sequence is embedded inside an expression vector.

According to one embodiment, the aforementioned sequence has a greater/smaller length than those tested up to now or chemically modified (m6A, w).

Another object is a pharmaceutical composition comprising at least one RNA sequence selected from those mentioned above, preferably from SINEUP_CHD8_003 and SINEUP CHD2 007.

Preferably, said pharmaceutical composition further comprises at least one pharmaceutically acceptable excipient.

Another object is a RNA or coding DNA sequence selected from those described above or SINEUP_CHD8_003, SINEUP_CHD2_007 or a combination thereof, or pharmaceutical composition as described above, for use in the treatment of a neurological disorder.

Another object is a RNA or coding DNA sequence selected from those listed above or, SINEUP_CHD8_003, SINEUP_CHD2_007 or a combination thereof, or pharmaceutical composition as described above, for use in the prevention of a neurological disorder. Preferably, said neurological disorder is an autism spectrum disorder or autism and/or epilepsy.

Another object is a RNA or coding DNA sequence selected from those listed above or SINEUP_CHD8_003, SINEUP_CHD2_007 or a combination thereof, or pharmaceutical composition described above, for use in a method directed to increase the expression of CHD8 and CHD2 proteins.

Another object is a DNA sequence encoding RNA sequence chosen from:
SINEUP_CHD8_001 having nucleotide sequence GCCATCTTGGGAAAGTAATGGAGGGTACTTCTCCAAGGTCTAGG,
SINEUP CHD2 006 having nucleotide sequence TCATCTTTTAATTCTTAAATATTAAGGGGGAGGGGGAATCTGTG, or a combination thereof.

Preferably, said sequence is embedded within an expression vector.

According to an embodiment of the above sequence, but of a greater/smaller length than those tested up to now or chemically modified (m6A, w).

Another object is a pharmaceutical composition comprising at least one RNA encoding DNA sequence selected from SINEUP_CHD8_001, SINEUP_CHD2_006.

Preferably, said composition, further comprising at least one pharmaceutically acceptable excipient.

Another object is a DNA sequence encoding RNA selected from SINEUP_CHD8_001, SINEUP_CHD2_006, or a combination thereof, or a pharmaceutical composition as described above, for use in the treatment of a neurological disorder.

Another object is a DNA sequence encoding RNA selected from SINEUP_CHD8_001, SINEUP_CHD2_006, or a combination thereof, or a pharmaceutical composition according to as described above, for use in the prevention of a neurological disorder.

Preferably the use in said neurological disorder is an autism spectrum disorder or autism and/or epilepsy.

Another object is a DNA sequence encoding RNA selected from SINEUP_CHD8_001, SINEUP_CHD2_006, or a combination thereof, or a pharmaceutical composition as described above, for use in a method directed to increase the expression of CHD8 and CHD2 proteins.

1. RNA or DNA sequence encoding a RNA sequence chosen from:
SINEUP_CHD8 003 having nucleotide sequence CCATTATCTGAGCCTGCTGTACAGAGGACAGAGTCACTGGCTGG,
SINEUP_CHD8_010 having nucleotide sequence CCAUUCCUGUCUUUCCCCCCGAAUGAGGAGCAGAGCUUGCUGGU,
SINEUP_CHD8_011 having nucleotide sequence GCAUGACUUCCACAUCUGAAUUAUCAGAUGAGGUAUUACGUUUU,
SINEUP CHD8 012 having nucleotide sequence GCAUGGAAGGCAAAGUCUCGCCAUCUGGUUCUUGCACUGGUUCA,
SINEUP CHD8 013 having nucleotide sequence GCAUAGAAAGCACUUUGUCUACAAUGGCUGCAUCUUCUUCACUG,
SINEUP CHD8 014 having nucleotide sequence UCAUCUGAGCCAUUUUGGUUUUGAAGCGCUUUAAUUUUUGAUGUAUCCUC UUA,
SINEUP CHD8 015 having nucleotide sequence UCAUUUCUGUCCAUGUAUUAAAUUCUCGCUCCCAGUUAGUAAUU,
SINEUP_CHD8 016 having nucleotide sequence ACAUUUCAUACUGUUGAAUCAUCUGCCUGCUGGCCAGACUGCCA,
SINEUP_CHD8 018 having nucleotide sequence GCAUCAUUGGCUUAAGAAUGGCCUGUAGCUUUUGAACCUGUUCC,
SINEUP_CHD8 019 having nucleotide sequence CCAUCAUUGUGUUAAGUAGAUUAGGCAUGUUGGUAUGACCUGCC,
SINEUP_CHD2_007 having nucleotide sequence ACATCTTCTTCACATCAGCTATCCGTTCCTTCTTAGAGGCTGGC,
SINEUP CHD2 010 having nucleotide sequence UCAUUUCAAUGAGAUCAUCUGAGUCAGUCUCAAAGUCAUCAUCU,
SINEUP_CHD2_011 having nucleotide sequence CCAUCCAUUUACAUAGAUAUUCGGGCUCAUUUGAGGGUGCCGGC,
SINEUP CHD2 012 having nucleotide sequence CCAUUUCAUCAGCAAGGAUUACACUAUUAUUUUUGCACCAGGAA,
SINEUP CHD2 013 having nucleotide sequence UCAUCAUCUUUUAAUUCUUAAAUAUUAAGGGGGAGGGGGAAUCU,
or a combination thereof,
wherein said sequence is embedded inside an expression vector.

2. RNA or DNA sequence according to claim 1, but of greater/smaller length than those hitherto tested or chemically modified (m6A, w).

3. Pharmaceutical composition comprising at least one RNA sequence selected from those of claim 1 or SINEUP_CHD8_003 and SINEUP_CHD2_007.

4. Pharmaceutical composition according to claim 3, further comprising at least one pharmaceutically acceptable excipient.

5. RNA or coding DNA sequence selected from those of claim 1 or SINEUP_CHD8_003, SINEUP CHD2_007 or a combination thereof, or pharmaceutical composition according to any one of claims 3 to 4, for use in the treatment of a neurological disorder.

6. RNA or coding DNA sequence selected from those of claim 1 or SINEUP_CHD8_003, SINEUP_CHD2_007 or a combination thereof, or pharmaceutical composition according to any one of claims 3 to 4, for use in the prevention of a neurological disorder.

7. RNA or coding DNA sequence according to any one of claims 5 to 6, for use where said neurological disorder is an autism spectrum disorder or autism and/or epilepsy.

8. RNA or coding DNA sequence selected from those of claim 1 or SINEUP_CHD8_003, SINEUP_CHD2_007 or a combination thereof, or pharmaceutical composition according to any one of claims 3 to 4, for use in a method aimed at increasing protein expression CHD8 and CHD2.

9. DNA sequence encoding RNA sequence chosen from:
SINEUP_CHD8_001 having nucleotide sequence GCCATCTTGGGAAAGTAATGGAGGGTACTTCTCCAAGGTCTAGG,
SINEUP CHD2 006 having nucleotide sequence TCATCTTTTAATTCTTAAATATTAAGGGGGAGGGGGAATCTGTG,
or a combination thereof,
wherein said sequence is embedded inside an expression vector.

10. DNA sequence encoding RNA sequence according to claim 9, but of greater/smaller length than those hitherto tested or chemically modified (m6A, w).

11. Pharmaceutical composition comprising at least one DNA sequence encoding RNA sequence selected from SINEUP_CHD8_001, SINEUP_CHD2_006.

12. Pharmaceutical composition according to claim 11, further comprising at least one pharmaceutically acceptable excipient.

13. DNA sequence encoding RNA selected from SINEUP_CHD8_001, SINEUP_CHD2_006, or a combination thereof, or pharmaceutical composition according to any one of claims 11 to 12, for use in the treatment of a neurological disorder.

14. DNA sequence encoding RNA selected from SINEUP_CHD8_001, SINEUP_CHD2_006, or a combination thereof, or pharmaceutical composition according to any one of claims 11 to 12, for use in the prevention of a neurological disorder.

15. DNA sequence encoding RNA according to any one of claims 13 to 14, for use where said neurological disorder is an autism spectrum disorder or autism and/or epilepsy.

16. DNA sequence encoding RNA selected from SINEUP_CHD8_001, SINEUP_CHD2_006, or a combination thereof, or pharmaceutical composition according to any one of claims 11 to 12, for use in a method aimed at increasing the expression of CHD8 and CHD2 proteins.


SEQUENCE LISTING (21 sequences)

SEQ ID NO: 1 - DNA, 44 bp, synthetic construct
  source 1..44: mol_type = other DNA
ccattatctgagcctgctgtacagaggacagagtcactggctgg

SEQ ID NO: 2 - RNA, 44 nt, synthetic construct
  source 1..44: mol_type = other RNA
ccattcctgtctttccccccgaatgaggagcagagcttgctggt

SEQ ID NO: 3 - RNA, 44 nt, synthetic construct
  source 1..44: mol_type = other RNA
gcatgacttccacatctgaattatcagatgaggtattacgtttt

SEQ ID NO: 4 - RNA, 44 nt, synthetic construct
  source 1..44: mol_type = other RNA
gcatggaaggcaaagtctcgccatctggttcttgcactggttca

SEQ ID NO: 5 - RNA, 44 nt, synthetic construct
  source 1..44: mol_type = other RNA
gcatagaaagcactttgtctacaatggctgcatcttcttcactg

SEQ ID NO: 6 - RNA, 53 nt, synthetic construct
  source 1..53: mol_type = other RNA
tcatctgagccattttggttttgaagcgctttaatttttgatgtatcctctta

SEQ ID NO: 7 - RNA, 44 nt, synthetic construct
  source 1..44: mol_type = other RNA
tcatttctgtccatgtattaaattctcgctcccagttagtaatt

SEQ ID NO: 8 - RNA, 44 nt, synthetic construct
  source 1..44: mol_type = other RNA
acatttcatactgttgaatcatctgcctgctggccagactgcca

SEQ ID NO: 9 - RNA, 44 nt, synthetic construct
  source 1..44: mol_type = other RNA
gcatcattggcttaagaatggcctgtagcttttgaacctgttcc

SEQ ID NO: 10 - RNA, 44 nt, synthetic construct
  source 1..44: mol_type = other RNA
ccatcattgtgttaagtagattaggcatgttggtatgacctgcc

SEQ ID NO: 11 - DNA, 44 bp, synthetic construct
  source 1..44: mol_type = other DNA
acatcttcttcacatcagctatccgttccttcttagaggctggc

SEQ ID NO: 12 - RNA, 44 nt, synthetic construct
  source 1..44: mol_type = other RNA
tcatttcaatgagatcatctgagtcagtctcaaagtcatcatct

SEQ ID NO: 13 - RNA, 44 nt, synthetic construct
  source 1..44: mol_type = other RNA
ccatccatttacatagatattcgggctcatttgagggtgccggc

SEQ ID NO: 14 - RNA, 44 nt, synthetic construct
  source 1..44: mol_type = other RNA
ccatttcatcagcaaggattacactattatttttgcaccaggaa

SEQ ID NO: 15 - RNA, 44 nt, synthetic construct
  source 1..44: mol_type = other RNA
tcatcatcttttaattcttaaatattaagggggagggggaatct

SEQ ID NO: 16 - DNA, 44 bp, synthetic construct
  source 1..44: mol_type = other DNA
gccatcttgggaaagtaatggagggtacttctccaaggtctagg

SEQ ID NO: 17 - DNA, 44 bp, synthetic construct
  source 1..44: mol_type = other DNA
tcatcttttaattcttaaatattaagggggagggggaatctgtg

SEQ ID NO: 18 - DNA, 25 bp, synthetic construct
  source 1..25: mol_type = other DNA
gagaatggaatcataacttacttga

SEQ ID NO: 19 - DNA, 25 bp, synthetic construct
  source 1..25: mol_type = other DNA
gcaaatgtgcaagcaagtaacacct

SEQ ID NO: 20 - DNA, 24 bp, synthetic construct
  source 1..24: mol_type = other DNA
cactggatatcactctttctttgc

SEQ ID NO: 21 - DNA, 22 bp, synthetic construct
  source 1..22: mol_type = other DNA
gtggtgtgtcatcaaagaggtc